(19) United States
(12) Patent Application Publication
(10) Pub. No.: US 2006/0147484 A1
(43) Pub. Date: Jul. 6, 2006

(54) Urushiol-induced contact dermatitis composition, method of use, and method of manufacture

(76) Inventor: Mark Anthony Hestand, Miami, OK (US)

Correspondence Address: MARTIN & ASSOCIATES, LLC, P O BOX 548, CARTHAGE, MO, 64836-0548, US

(21) Appl. No.: 11/027,965

(22) Filed: Jan. 3, 2005

(51) Int. Cl. A61K 33/14; A61K 8/02

(52) U.S. Cl. 424/401; 424/680

(57) ABSTRACT

A composition for treating urushiol-induced contact dermatitis includes an aqueous solution of water, a first surfactant, and a second surfactant in proportions that bind a sufficient number of the water molecules that when this aqueous solution is mixed with granulated salt, the majority of the salt is not dissolved but stays in its granular form in suspension in the aqueous solution. The resulting composition has excellent properties for treating urushiol-induced contact dermatitis.

### BACKGROUND OF THE INVENTION

1. Technical Field

This invention generally relates to topical skin treatments, and more specifically relates to treatments for urushiol-induced contact dermatitis.

2. Background Art

Throughout the world, many plants contain a toxin known as urushiol, which causes itching and discomfort for people who come into contact with the plant. Examples of such plants in North America include poison ivy, poison oak, and poison sumac. Experts estimate there are up to 55 million cases of urushiol-induced contact dermatitis annually in the United States.

Anyone who has been afflicted with urushiol-induced contact dermatitis can attest to the discomfort it causes, both in discoloration due to the red rash and blisters as well as the persistent itch. It sometimes takes two or three weeks to completely heal. Some products have been developed to wash off urushiol before it has a chance to bind to the skin. These products do nothing for urushiol-induced contact dermatitis when the urushiol has already bound to the skin.

One known product provides some relief of the symptoms of urushiol-induced contact dermatitis. This product is disclosed in U.S. patent application 2002/0183284 A1 entitled “Urushiol-induced Contact Dermatitis Solution”, filed on Jun. 22, 2002. Paragraph [0036] of this referenced patent application gives a composition for the solution that is the same as in the commercial product Zanfel, which is manufactured and marketed by Zanfel Laboratories, Inc. at PO Box 349, Morton, Ill. 61550. Zanfel is a trademark of Zanfel Laboratories, Inc. Zanfel is a composition that includes sodium lauroyl sarcosinate, which has been found to be somewhat effective in treating symptoms of urushiol-induced contact dermatitis.

Zanfel suffers from several problems. The composition of Zanfel makes it difficult to deliver a sufficient and consistent amount of the active ingredient to the urushiol that causes the itch and discomfort. Zanfel comes in a dry paste form that contains a large amount of polyethylene beads, which are chemically inert but are present as an abrasive for scrubbing the skin. The relatively large amount of polyethylene beads in Zanfel leaves little room for active ingredients needed to combat the urushiol. In addition, the application process for Zanfel does not allow for regulating or delivering a consistent concentration of active ingredient during treatment. The consumer starts by adding water to the dry paste to make a wet paste. The consumer then scrubs the affected area with the paste. As the paste dries, the consumer has to add additional water to be able to continue to wash the affected area. But how much water should the consumer initially add? The consumer's perception of the terms “paste” and “dry” leave a huge dosage range of concentration of the active ingredient in Zanfel that is actually delivered to the affected area. As a result, it is difficult to achieve consistent results with Zanfel due to the variables in its application that result in varying levels of active ingredient.

In addition, Zanfel is very expensive, which means many people who contract urushiol-induced contact dermatitis will often not want to spend the money required for treatment using Zanfel. The cost for one ounce of Zanfel is so high that it often rivals the expense of going to the doctor and getting a prescription for treating urushiol-induced contact dermatitis. As a result, Zanfel does not provide a significant alternative in cost to the physician/prescription method of treating urushiol-induced contact dermatitis.

The prescription medications that are available for treating urushiol-induced contact dermatitis are usually anti-inflammatory medications that often carry the risk of considerable side effects or complications. Without a way to provide a more effective, affordable, over-the-counter alternative to potentially harmful anti-inflammatory prescription medications, many people afflicted with poison ivy, poison oak, or poison sumac exposure will continue to suffer from symptoms of urushiol-induced contact dermatitis.

### DISCLOSURE OF INVENTION

According to the preferred embodiments, a composition for treating urushiol-induced contact dermatitis includes an aqueous solution of water, a first surfactant, and a second surfactant in proportions that bind a sufficient number of the water molecules that when this aqueous solution is mixed with granulated salt, the majority of the salt is not dissolved but stays in its granular form in suspension in the aqueous solution. The resulting composition has excellent properties for treating urushiol-induced contact dermatitis.

The foregoing and other features and advantages of the invention will be apparent from the following more particular description of preferred embodiments of the invention, as illustrated in the accompanying drawings.

### BRIEF DESCRIPTION OF DRAWINGS

The preferred embodiments of the present invention will hereinafter be described in conjunction with the appended drawings, where like designations denote like elements, and:

FIG. 1 is a flow diagram of a method for manufacturing the composition for treating urushiol-induced contact dermatitis in accordance with the preferred embodiments; and

FIG. 2 is a flow diagram of a method for treating affected areas of the body with the composition in accordance with the preferred embodiments.

### BEST MODE FOR CARRYING OUT THE INVENTION

The composition of the preferred embodiments provides for effective treatment of urushiol-induced contact dermatitis. An aqueous solution of water, a first surfactant, and a second surfactant are combined in proportions that bind a majority of the water molecules. This aqueous solution is then mixed with granular salt, which causes a majority of the salt to remain in suspension in the composition in granular form, rather than dissolving, due to the unavailability of the bound water molecules.

The composition of the preferred embodiments preferably includes an aqueous solution of water, a first surfactant, and a second surfactant in specified proportions that achieve the binding of a majority of the water molecules. Note that the percentages herein are specified as a percentage based on volume of the ingredients, not weight. One suitable composition within the scope of the preferred embodiments includes 20-80 percent water, 5-20 percent first surfactant, and 15-60 percent second surfactant. A preferred composition within the scope of the preferred embodiments includes 37-71 percent water, 9-18 percent first surfactant, and 20-45 percent second surfactant. The most preferred composition within the scope of the preferred embodiments includes 47-58 percent water, 12-15 percent first surfactant, and 30-38 percent second surfactant. Once the aqueous solution is mixed in the desired proportions, a predetermined quantity of granulated salt is mixed with the aqueous solution. In one suitable implementation, the quantity of granulated salt is sufficient to achieve a concentration of 10-50 percent granulated salt by volume. In a preferred implementation, the quantity of granulated salt is sufficient to achieve a concentration of 20-40 percent granulated salt by volume. In a most preferred implementation, the quantity of granulated salt is sufficient to achieve a concentration of approximately 25-30 percent granulated salt by volume.

The specific ingredients used in the composition may vary within the scope of the preferred embodiments. The water is preferably distilled, deionized, or distilled and deionized water, but could also include other types of water, including filtered, bottled and tap water. The first surfactant is preferably a soap base that includes any compounds known as sulfate or sulphonic surfactants. The first surfactant may include any one or more of the following: sodium laureth sulfate, sodium lauryl sulfate, dodecylsulfate and ammonium lauryl sulfate. In the most preferred implementation, the first surfactant is sodium laureth sulfate in a soap base marketed as Super Concentrate by Soapcrafters at 2944 S. West Temple, Salt Lake City, Utah 84115. Note that Super Concentrate includes other ingredients such as cocamidopropyl Betaine, Cocamide DEA, Cocamidopropyl hydroxysultaine, Glydent Antimicrobial, and EDTA-4NA organic chelating agent. The main benefit of using the Super Concentrate formulation is the presence of these additional ingredients that do not inhibit the delivery of the first surfactant to the affected area. Further, the emulsifiers, chelating agent, and stabilizers create a desirable carrier for the composition and add to the shelf life of the composition. Finally, the Glydent anti-microbial allows the resulting composition to meet the requirements of the Food and Drug Administration (FDA) that the composition does not host nor grow harmful microbes.

The second surfactant is preferably a sarcosine or a companion salt compound known as a sarcosinate. The second surfactant may include any one or more of the following: sodium N-lauroyl sarcosinate, sodium N-cocoyl sarcosinate, and sodium N-myristoyl sarcosinate. In the most preferred implementation the second surfactant is sodium N-cocoyl sarcosinate in a product known as CS-30, which is available from Croda Inc., 300-A Colombus Circle, Edison, N.J. 08837-3907.

The salt is preferably granulated sodium chloride, but may also be potassium chloride, magnesium sulfate, and sea salt. The size of the salt granules affects how well the composition of the preferred embodiments works, and clinical trials have shown that fine-ground sodium chloride works well. In the most preferred implementation, the size of the salt granules ranges from medium course sand to very fine silt as measured on the Udden-Wentworth scale. Granulated salt is available from numerous commercial sources, including Morton International Inc. in Chicago, Ill.

Other ingredients may be added to the composition to give it more desirable effects. For example, an organic acid such as sodium bisulphate, hydrochloric acid, or sulfuric acid could be added to lower the pH of the composition to a level that is more compatible with human skin, such as a range of 6-8 pH. An anti-microbial could be added to avoid growth of microbes in the composition. An emulsifier could also be added to keep the various ingredients in an emulsified mixture. A skin conditioner could also be added to help keep the skin soft after treatment. The addition of these and other ingredients to enhance the composition is within the scope of the preferred embodiments.

A significant feature of the composition of the preferred embodiments is the presence of the granulated salt in the composition. Note that the first and second surfactants preferably bind a majority of the water molecules, which means a majority of the granulated salt remains suspended in granule form instead of dissolving into the aqueous solution. The granulated salt performs two functions. First, it acts as an abrasive to help expose urushiol in the affected area as the area is scrubbed with the composition. Second, the salt causes an osmotic effect that causes the affected area to “weep”, meaning that the salt draws out the fluid from the affected area. Such fluid may contain additional urushiol that is not readily available at the surface of the skin. In the commercial product Zanfel, polyethylene granules are used as an inert material that provides abrasive particles that help to scrub the affected area. The salt in the composition of the preferred embodiments is far superior to polyethylene granules because the salt provides the abrasive particles while at the same time providing osmotic properties that help to cleanse the affected area. For this reason, the ability of the composition of the preferred embodiments to treat urushiol-induced contact dermatitis is enhanced when compared to known products.

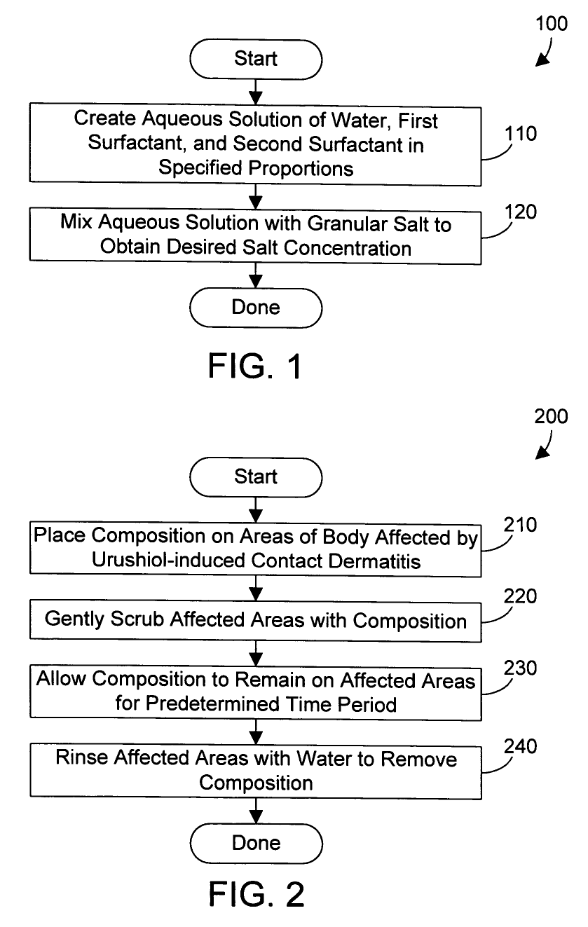

The preferred embodiments include a method for manufacturing the composition. Referring to FIG. 1, a method 100 in accordance with the preferred embodiments for manufacturing the composition starts by creating an aqueous solution of water, first surfactant and second surfactant in specified proportions (step 110). The specified proportions in step 110 are such that the majority of the water molecules are bound to one or both of the surfactants. Next, the aqueous solution is mixed with granular salt to obtain the desired salt concentration (step 120). It is significant to note that because a majority of the water molecules in the aqueous solution are bound to one or both of the surfactants, the majority of the granular salt will remain in its granular form when mixed with the aqueous solution, which will cause the granular salt to remain in suspension in the composition instead of being dissolved. As stated above, the granulated salt in suspension serves two important roles, one physical and one chemical. The physical role of the granulated salt is the role of abrasive particles that help to expose an affected area. The chemical role of the granulated salt is an osmotic agent that helps to draw additional fluid from under the surface of the skin. This additional fluid may contain urushiol that is not readily available on the surface of the skin. This dual function of the granulated salt provides enhanced cleaning properties when treating skin that is affected by urushiol-induced contact dermatitis.

The preferred embodiments also include a method for treating urushiol-induced contact dermatitis. Referring to FIG. 2, a method 200 in accordance with the preferred embodiments starts by placing the composition on areas of the body affected by urushiol-induced contact dermatitis (step 210). The affected areas are gently scrubbed with the composition (step 220). During the scrubbing in step 220, the salt granules in the composition serve as abrasive particles that help to expose the urushiol and draw additional fluid out of the skin that may contain urushiol. The composition is then allowed to remain on the affected areas for a predetermined period of time (step 230). In actual tests, it has been found that leaving the composition on the affected area for 30 seconds to one minute is generally effective. During the time that the composition is allowed to remain on the affected areas in step 230, the urushiol will continue to bind with one or both of the surfactants. The final step is to rinse the affected areas with water to remove the composition (step 240). Note that the composition will attach to urushiol molecules during steps 210, 220 and 230. As a result, when the composition is rinsed off in step 240, much of the urushiol is rinsed away with the used composition.

The composition of the preferred embodiments not only provides relief from the itch associated with urushiol-induced contact dermatitis, but also binds with the urushiol itself to rid the body of the toxin that causes the inflammation, red skin, itch, pustules, etc. In other words, the composition of the preferred embodiments not only treats the itch symptom, it also rids the affected area of the urushiol that is the underlying cause of the itch, redness, and other symptoms. For this reason, the composition of the preferred embodiments is far superior to some known products, such as cortisone cream, that only treat the itch caused by urushiol.

Best Mode

While the composition, method of manufacture, and method of treating urushiol-induced dermatitis are enabled above, a best mode is now presented. In the most preferred formulation, the composition includes 50% water, 38% sodium N-cocoyl sarcosinate, and 12% Super Concentrate soap referenced above. This aqueous solution is mixed with fine-ground granulated salt at a ratio of 75% aqueous solution to 25% granulated salt by volume. These percentage concentrations by volume may be achieved using the following formula:
4 fluid oz. distilled water
3 fluid oz. CS-30
1 fluid oz. Super Concentrate soap
sodium bisulphate in sufficient quantity to reduce the pH to the 6.0-7.0 range
   This aqueous solution is mixed thoroughly. Next the salt is added:
very fine granulated sodium chloride mixed at a rate of approximately one part sodium chloride to three parts aqueous solution, by volume
   The resulting composition has been tested, and has proven to provide substantial relief of the symptoms of urushiol-induced contact dermatitis, sometimes in only a single treatment. Because the composition is in a liquid gel form and does not require the consumer to mix it with water during treatment, the amount of active ingredients that remove urushiol is better controlled than using drier compounds that are mixed with water by the consumer.

One skilled in the art will appreciate that many variations are possible within the scope of the present invention. Thus, while the invention has been particularly shown and described with reference to preferred embodiments thereof, it will be understood by those skilled in the art that these and other changes in form and details may be made therein without departing from the spirit and scope of the invention.

What is claimed is:

1. A composition for treating urushiol-induced contact dermatitis, the composition comprising:
granulated salt; and
an aqueous solution of water, a first surfactant, and a second surfactant in proportions that bind a sufficient number of molecules of the water that at least half of the granulated salt remains in suspension in the aqueous solution without being dissolved in the aqueous solution.

2. The composition of claim 1 wherein the proportions include:
20-80 percent water;
5-20 percent first surfactant; and
15-60 percent second surfactant.

3. The composition of claim 1 wherein the proportions include:
37-71 percent water;
9-18 percent first surfactant; and
20-45 percent second surfactant.

4. The composition of claim 1 wherein the proportions include:
47-58 percent water;
12-15 percent first surfactant; and
30-38 percent second surfactant.

5. The composition of claim 1 wherein the granulated salt and aqueous solution are mixed to achieve a concentration of 10-50 percent granulated salt by volume.

6. The composition of claim 1 wherein the granulated salt and aqueous solution are mixed to achieve a concentration of 20-40 percent granulated salt by volume.

7. The composition of claim 1 wherein the granulated salt and aqueous solution are mixed to achieve a concentration of approximately 25-30 percent granulated salt by volume.

8. The composition of claim 1 wherein:
the salt is selected from the group consisting of sodium chloride, potassium chloride, magnesium sulfate, and sea salt;
the first surfactant is selected from the group consisting of sodium laureth sulfate, sodium lauryl sulfate, dodecylsulfate, and ammonium lauryl sulfate; and
the second surfactant is selected from the group consisting of sodium N-lauroyl sarcosinate, sodium N-cocoyl sarcosinate, and sodium N-myristoyl sarcosinate.

9. A composition for treating urushiol-induced contact dermatitis, the composition comprising:
(A) an aqueous solution comprising:
47-58 percent water;
12-15 percent sodium laureth sulfate;
30-38 percent sodium cocoyl sarcosinate;
(B) granulated salt mixed with the aqueous solution to achieve a concentration of approximately 25-30 percent granulated salt by volume.

10. A method for manufacturing a composition for treating urushiol-induced contact dermatitis, the method comprising the steps of:
(A) providing a predetermined quantity of granulated salt;
(B) creating an aqueous solution of water, a first surfactant, and a second surfactant in proportions that bind a sufficient number of molecules of the water that at least half of the predetermined quantity of granulated salt remains in suspension in the aqueous solution without being dissolved in the aqueous solution when the predetermined quantity of salt is mixed with the aqueous solution; and
(C) mixing the predetermined quantity of granulated salt with the aqueous solution.

11. The method of claim 10 wherein the proportions include:
20-80 percent water;
5-20 percent first surfactant; and
15-60 percent second surfactant.

12. The method of claim 10 wherein the proportions include:
37-71 percent water;
9-18 percent first surfactant; and
20-45 percent second surfactant.

13. The method of claim 10 wherein the proportions include:
47-58 percent water;
12-15 percent first surfactant; and
30-38 percent second surfactant.

14. The method of claim 10 wherein the granulated salt and aqueous solution are mixed in step (C) to achieve a concentration of 10-50 percent granulated salt by volume.

15. The method of claim 10 wherein the granulated salt and aqueous solution are mixed in step (C) to achieve a concentration of 20-40 percent granulated salt by volume.

16. The method of claim 10 wherein the granulated salt and aqueous solution are mixed in step (C) to achieve a concentration of approximately 25-30 percent granulated salt by volume.

17. The method of claim 10 wherein:
the salt is selected from the group consisting of sodium chloride, potassium chloride, magnesium sulfate, and sea salt;
the first surfactant is selected from the group consisting of sodium laureth sulfate, sodium lauryl sulfate, dodecylsulfate, and ammonium lauryl sulfate; and
the second surfactant is selected from the group consisting of sodium N-lauroyl sarcosinate, sodium N-cocoyl sarcosinate, and sodium N-myristoyl sarcosinate.

18. A method for manufacturing a composition for treating urushiol-induced contact dermatitis, the method comprising the steps of:
(A) creating an aqueous solution in the following proportions:
47-58 percent water;
12-15 percent sodium laureth sulfate;
30-38 percent sodium cocoyl sarcosinate;
(B) mixing granulated salt with the aqueous solution to achieve a concentration of approximately 25-30 percent granulated salt by volume.

19. A method for treating urushiol-induced contact dermatitis, the method comprising:
(A) placing a composition on affected areas of a body, the composition comprising:
granulated salt; and
an aqueous solution of water, first surfactant, and second surfactant in proportions that bind at least half of the molecules of water such that at least half of the granulated salt remains in suspension in the aqueous solution without being dissolved in the aqueous solution;
(B) scrubbing the affected areas with the composition;
(C) allowing the composition to remain on the affected areas for a predetermined time period; and
(D) rinsing the affected areas with water to remove the composition from the affected areas.

20. The method of claim 19 wherein the proportions include:
20-80 percent water;
5-20 percent first surfactant; and
15-60 percent second surfactant.

21. The method of claim 19 wherein the proportions include:
37-71 percent water;
9-18 percent first surfactant; and
20-45 percent second surfactant.

22. The method of claim 19 wherein the proportions include:
47-58 percent water;
12-15 percent first surfactant; and
30-38 percent second surfactant.

23. The method of claim 19 wherein the granulated salt and aqueous solution are mixed in step (C) to achieve a concentration of 10-50 percent granulated salt by volume.

24. The method of claim 19 wherein the granulated salt and aqueous solution are mixed in step (C) to achieve a concentration of 20-40 percent granulated salt by volume.

25. The method of claim 19 wherein the granulated salt and aqueous solution are mixed in step (C) to achieve a concentration of approximately 25-30 percent granulated salt by volume.

26. The method of claim 19 wherein:
the salt is selected from the group consisting of sodium chloride, potassium chloride, magnesium sulfate, and sea salt;
the first surfactant is selected from the group consisting of sodium laureth sulfate, sodium lauryl sulfate, dodecylsulfate, and ammonium lauryl sulfate; and
the second surfactant is selected from the group consisting of sodium N-lauroyl sarcosinate, sodium N-cocoyl sarcosinate, and sodium N-myristoyl sarcosinate.

27. A method for treating urushiol-induced contact dermatitis, the method comprising:
(A) placing a composition on affected areas of a body, the composition comprising:
(A1) an aqueous solution in the following proportions:
47-58 percent water;
12-15 percent sodium laureth sulfate;
30-38 percent sodium cocoyl sarcosinate;
(A2) granulated salt in sufficient quantity to achieve a concentration of approximately 25-30 percent granulated salt by volume when mixed with the aqueous solution;
(B) scrubbing the affected areas with the composition;
(C) allowing the composition to remain on the affected areas for a predetermined time period; and
(D) rinsing the affected areas with water to remove the composition from the affected areas.
